(19) United States
(12) Patent Application Publication
(10) Pub. No.: US 2015/0105795 A1
(43) Pub. Date: Apr. 16, 2015

(54) Deployable and Multi-Sectional Hearing Implant Electrode

(71) Applicant: MED-EL Elektromedizinische Geraete GmbH, Innsbruck (AT)

(72) Inventors: Thomas Lenarz, Hannover (DE); Anandhan Dhanasingh, Innsbruck (AT); Claude Jolly, Innsbruck (AT); Ingeborg Hochmair, Axams (AT); Anita Ridl, Kematen (AT)

(21) Appl. No.: 14/514,963

(22) Filed: Oct. 15, 2014

Related U.S. Application Data
(60) Provisional application No. 61/890,923, filed on Oct. 15, 2013.

(51) Int. Cl. A61N 1/05

(52) U.S. Cl. CPC A61N 1/0541
USPC 606/129; 607/137

(57) ABSTRACT

An implantable electrode is described for a cochlear implant patient with a malformed common cavity cochlea having a single internal cavity defined by an outer cavity wall or patients having an incomplete partition or conventional spiral-shaped cochlea. An intra-cochlear electrode array is configured to be inserted into the cochlea through a single cochleostomy opening. Outer array branches are closable about a center axis and the electrode array is configured to be closed into a single tube including the array branches for insertion through the single cochleostomy opening into the internal chamber of the cochlea. The electrode array opens within the internal cavity after insertion into the cochlea so that the array branches move away from the center axis to lie with their outer surfaces against the outer cavity wall to deliver the electrical stimulation signals through the stimulation contacts to adjacent neural tissue for auditory perception by the patient.

This application claims priority from U.S. Provisional Patent Application 61/890,923, filed Oct. 15, 2013, which is incorporated herein by reference in its entirety.

### FIELD OF THE INVENTION

The present invention relates to medical implants, and more specifically to an implantable electrode for use in cochlear implant systems in patients having a malformed cochlea.

### BACKGROUND ART

A normal ear transmits sounds as shown in FIG. 1 through the outer ear 101 to the tympanic membrane (eardrum) 102, which moves the bones of the middle ear 103 (malleus, incus, and stapes), which in turn vibrate the oval window and round window openings of the cochlea 104. The cochlea 104 is a long narrow duct wound spirally about its axis for approximately two and a half turns. The cochlea 104 includes an upper channel known as the scala vestibuli and a lower channel known as the scala tympani, which are connected by the cochlear duct. The scala tympani forms an upright spiraling cone with a center called the modiolar where the spiral ganglion cells of the acoustic nerve 113 reside. In response to received sounds transmitted by the middle ear 103, the fluid-filled cochlea 104 functions as a transducer to generate electric pulses that are transmitted to the cochlear nerve 113, and ultimately to the brain.

Hearing is impaired when there are problems in the ability to transduce external sounds into meaningful action potentials along the neural substrate of the cochlea. In such cases a cochlear implant is an auditory prosthesis which uses an implanted stimulation electrode to bypass the acoustic transducing mechanism of the ear and instead stimulate auditory nerve tissue directly with small currents delivered by multiple electrode contacts distributed along the electrode.

FIG. 1 also shows some components of a typical cochlear implant system which includes an external microphone that provides an audio signal input to an external signal processing stage 111 where various signal processing schemes can be implemented. The processed signal is then converted into a digital data format, such as a sequence of data frames, for transmission into the implant stimulator 108. Besides extracting the audio information, the implant stimulator 108 also performs additional signal processing such as error correction, pulse formation, etc., and produces a stimulation pattern (based on the extracted audio information) that is sent through connected wires 109 to an implanted electrode carrier 110. Typically, this electrode carrier 110 includes multiple electrodes on its surface that provide selective stimulation of the cochlea 104.

Cochlear implant systems need to deliver electrical power from outside the body through the skin to satisfy the power requirements of the implanted portion of the system. As shown in FIG. 1, an external transmitter coil 107 (coupled to the external signal processor 111) is placed on the skin adjacent to a subcutaneous receiver coil connected to the implant stimulator 108. Often, a magnet in the external coil structure interacts with a corresponding magnet in the subcutaneous secondary coil structure. This arrangement inductively couples a radio frequency (rf) electrical signal to the implant stimulator 108. The implant stimulator 108 is able to extract from the rf signal both the audio information for the implanted portion of the system and a power component to power the implanted system.

In some persons, the cochlear shape fails to develop properly and various malformation conditions can occur such as those shown in FIG. 2: cochlear aplasia, cochlear hypoplasia, common cavity (CC) malformation, and incomplete partitioning. Specifically in a common cavity malformation the cochlea and the vestibule are represented by a single chamber. This structure may have cochlear and vestibular neural structures, but it completely lacks inter-scala separation (no basilar membrane), no modiolus trunk, and it appears as a single cavity. The neural structures are believed to be present at the bony capsule defining the outer cavity wall. The specific size of the cavity can vary significantly and can be measured using medical imaging.

Placing an electrode inside this common cavity is not straightforward and needs utmost care to ensure that the stimulation contacts are either touching or very close to the outer wall of the cavity. The current technique involves making two cochleostomy openings in the outer surface of the cochlea for the electrode placement, which is undesirably traumatic.

FIG. 3A shows one approach wherein the electrode array 302 has an extended distal end. Two cochleostomies 304 are made in the outer surface of the cochlea 300, the electrode array 302 is inserted through one of the cochleostomies 304, and the distal tip of the electrode array 302 is retrieved and pulled through the other cochleostomy 304. The surgeon has to manipulate the electrode array 302 to attempt to place the stimulation contacts 303 against the outer wall 301 of the cavity, after which the final position of the electrode array 302 is fixed and the distal extension may be removed.

FIG. 3B shows another approach for electrode implantation in a common cavity, again requiring two cochleostomies 304 in the outer surface of the cochlea 300. Two separate electrode arrays 302 are used, one through each cochleostomy 304, and again considerable surgical skill is needed to manipulate the electrode arrays 302 to place their stimulation contacts 303 adjacent to the outer wall 301 of the cavity. Both techniques are highly traumatic in requiring two cochleostomies and both require considerable surgical skill to be effective.

### SUMMARY OF THE INVENTION

Embodiments of the present invention are directed to an implantable electrode for a cochlear implant patient with a malformed common cavity cochlea having a single internal cavity defined by an outer cavity wall. An extra-cochlear electrode lead contains signal wires for conducting electrical stimulation signals. An intra-cochlear electrode array is configured to be inserted into the cochlea through a single cochleostomy opening. Outer array branches are closable about a center axis and the electrode array is configured to be closed into a single tube including the array branches for insertion through the single cochleostomy opening into the internal chamber of the cochlea. The electrode array opens within the internal cavity after insertion into the cochlea so that the array branches move away from the center axis to lie with their outer surfaces against the outer cavity wall to deliver the electrical stimulation signals through the stimulation contacts to adjacent neural tissue for auditory perception by the patient.

An outer insertion tube is configured to contain the folded electrode array for insertion into the cochlea, and is retractable back through the cochleostomy opening after insertion of the electrode array into the cochlea to allow the array branches to open within the internal cavity of the cochlea. In specific embodiments the insertion tube may contain a longitudinal slit along its outer surface for removal of the insertion tube from the electrode lead after retraction. The insertion tube also may include one or depth indicator marks along its outer surface.

The electrode array may be configured to be closed into a closed umbrella shape with the array branches lying along the center axis for insertion into the cochlea and to be opened into an open umbrella shape after insertion into the cochlea. Or the electrode array may be configured to be closed into a closed inverted umbrella shape with the array branches lying along the center axis for insertion into the cochlea and to be opened into an open inverted umbrella shape after insertion into the cochlea. The outer surface of each array branch may include multiple stimulation contacts, which may be on inner and/or outer surfaces. The array branches may include a springy support wire within the array branches that biases the array branches open.

The electrode array may be made of radio-opaque material. And the array branches have different radio-opaque patterns and/or a distal tip of the electrode array may have a radio indicator mark.

There may be a central array trunk containing the signal wires against which the array branches are closed for insertion into the cochlea. Or there may be a central inflation balloon against which the array branches are closed for insertion into the cochlea. The inflation balloon then is filled and expands outward after insertion into the cochlea to move the array branches away from the center axis to place their outer surfaces against the outer cavity wall. The inflation balloon may be permanently connected to the array branches, or it may be configured to be deflated after being filled and to be removed from the cochlea via the single cochleostomy opening.

The electrode array may include a connector tip configured to hold together distal ends of the array branches, so that the array branches to move away from the center axis after the connector tip touches the outer cavity wall opposite the single cochleostomy opening during insertion of the electrode array into the cochlea. The connector tip may be permanently connected to the distal ends of the array branches, or it may be removable from the distal ends of the array branches after insertion of the electrode array into the cochlea.

Embodiments of the present invention also include a cochlear implant electrode for a cochlear implant patient with an incomplete partition or conventional spiral-shaped cochlea. An extra-cochlear electrode lead contains signal wires for conducting electrical stimulation signals. An intra-cochlear electrode array is configured to be inserted into the cochlea through a single cochleostomy opening, and it includes outer array branches that are closable about a center axis. The electrode array is configured to be closed into a single tube including the array branches for insertion through the single cochleostomy opening into the internal chamber of the cochlea, and configured to be opened within the cochlea after insertion so that the array branches move away from the center axis to lie with their outer surfaces against an modiolar wall of the cochlea to deliver the electrical stimulation signals through the stimulation contacts to adjacent neural tissue for auditory perception by the patient.

Embodiments also include a complete cochlear implant system having an electrode array according to any of the above.

### BRIEF DESCRIPTION OF THE DRAWINGS

FIG. 1 shows elements of a human ear having a typical cochlear implant system.

FIG. 2 illustrates various cochlear malformation shapes.

FIGS. 3 A-B show conventional electrode insertion into a common cavity cochlea using two cochleostomies.

FIGS. 4 A-B show a common cavity electrode having an umbrella shape according to an embodiment of the present invention.

FIGS. 5 A-E show various aspects of a common cavity electrode having an inverted umbrella shape according to an embodiment of the present invention.

FIGS. 6 A-B show an outer insertion tube around the electrode array according to an embodiment of the present invention.

FIGS. 7 A-B show electrode arrays according to embodiments of the present invention after insertion into the common cavity.

FIGS. 8 A-B show another embodiment of a common cavity electrode.

FIGS. 9 A-B show another embodiment of a common cavity electrode.

FIGS. 10 A-B show another embodiment of a common cavity electrode.

FIGS. 11 A-D show an embodiment of a cochlear implant electrode with foldable array branches for a normal cochlear anatomy or a cochlea with incomplete partition (e.g., Mondini dysplasia) with contacts to both sides.

FIGS. 12 A-B show another embodiment of a common cavity electrode.

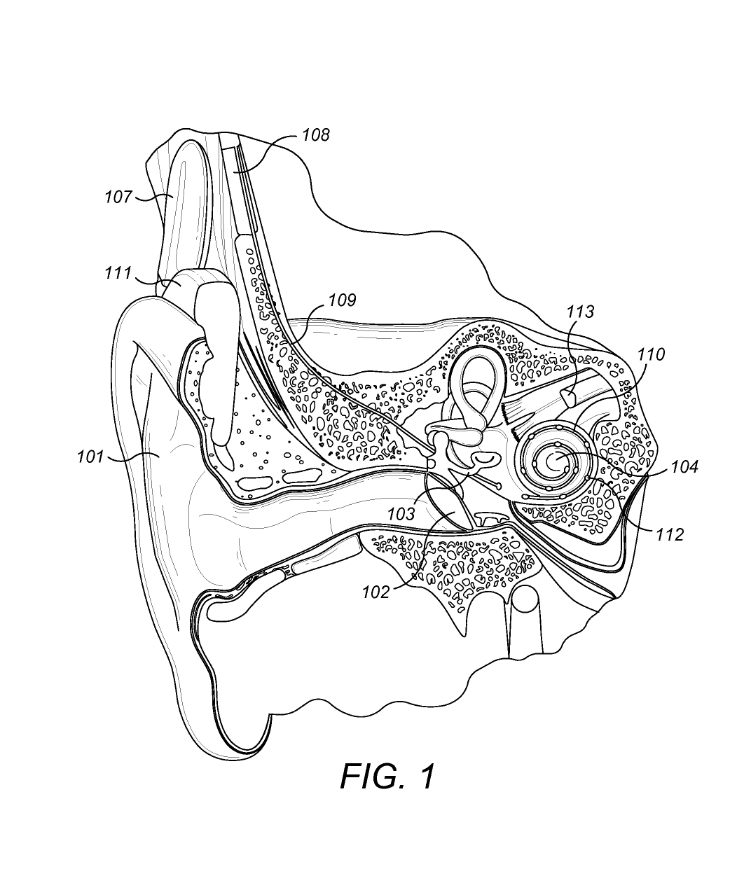

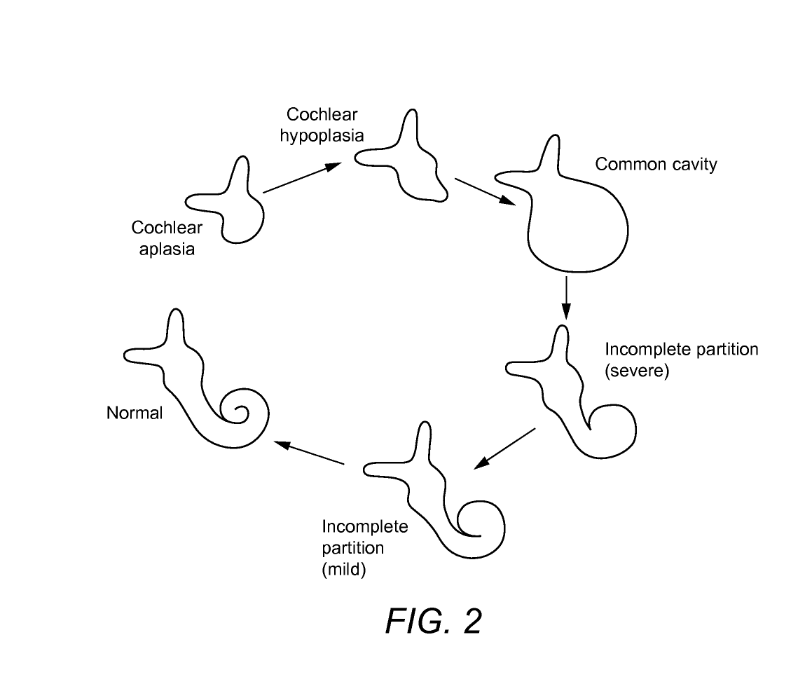

### DETAILED DESCRIPTION OF SPECIFIC EMBODIMENTS

Various embodiments of the present invention are directed to an implantable electrode for a common cavity cochlea having a foldable electrode array configured for insertion into the cochlea through a single cochleostomy. After entering the common cavity, the electrode array is unfolded to place array branches with the stimulating contacts adjacent to the outer cavity wall. Because the electrode is configured for insertion through a single cochleostomy opening rather than requiring two cochleostomies as in existing conventional arrangements, the amount of trauma to the cochlea is reduced and an easier surgical insertion process can be used. Further, the probability to make contact with neural elements is increased, which location and distribution inside malformed cochleae cannot be predicted by preoperative examinations.

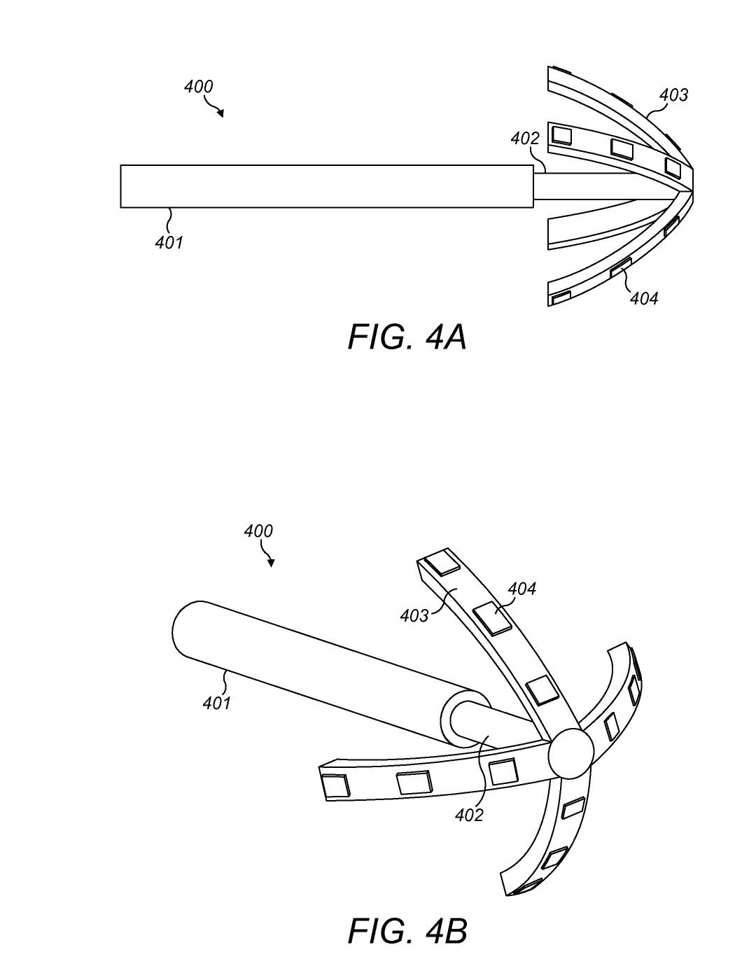

FIG. 4 A-B show a common cavity electrode 400 having an umbrella shape according to an embodiment of the present invention. The common cavity electrode 400 includes an extra-cochlear electrode lead 401 that contains the signal wires that conduct the electrical stimulation signals. An intra-cochlear electrode array includes a central array trunk 402 that is elongated along a center axis and that contains the signal wires. Multiple outer array branches 403 of flexible bio-compatible polymer material are foldable about the center axis. The array branches 403 as shown in FIG. 4 A-B are foldable into an umbrella shape to lie against the array trunk 402 for insertion into the cochlea. The array branches 403 have multiple stimulation contacts 404, which may specifically be on the inner and/or outer surfaces of the array branches 403. The electrode array branches 403 and/or the array trunk 402 may be made of radio-opaque material, and there may be a radio indicator mark on the distal tip of the array trunk 402 for medical imaging systems. Alternatively, only portions of the array branches 403 may be made of radio-opaque material such that they have different radio-opaque patterns and consequently they differ from each other on the image, e.g. an X-ray image. The knowledge about the exact placement of the individual array branches 403 may be helpful for later fitting of the device to the patient's needs and to avoid misplacement. In further embodiments there may also be stimulation contacts on the array trunk 402.

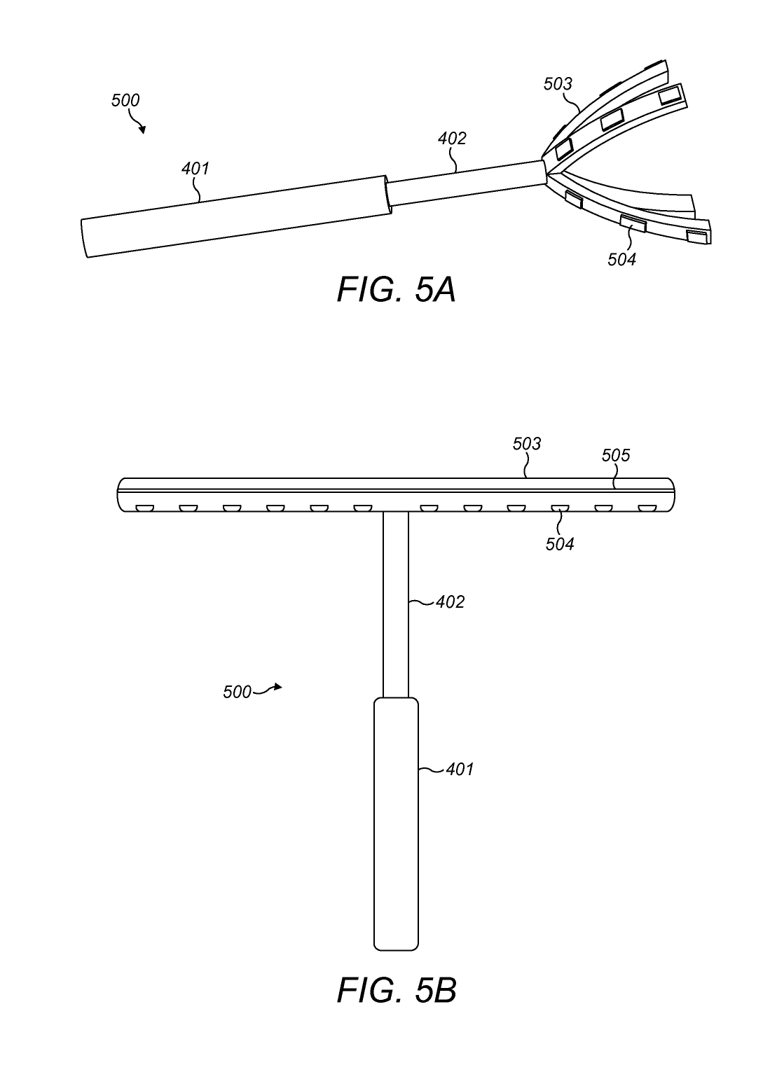

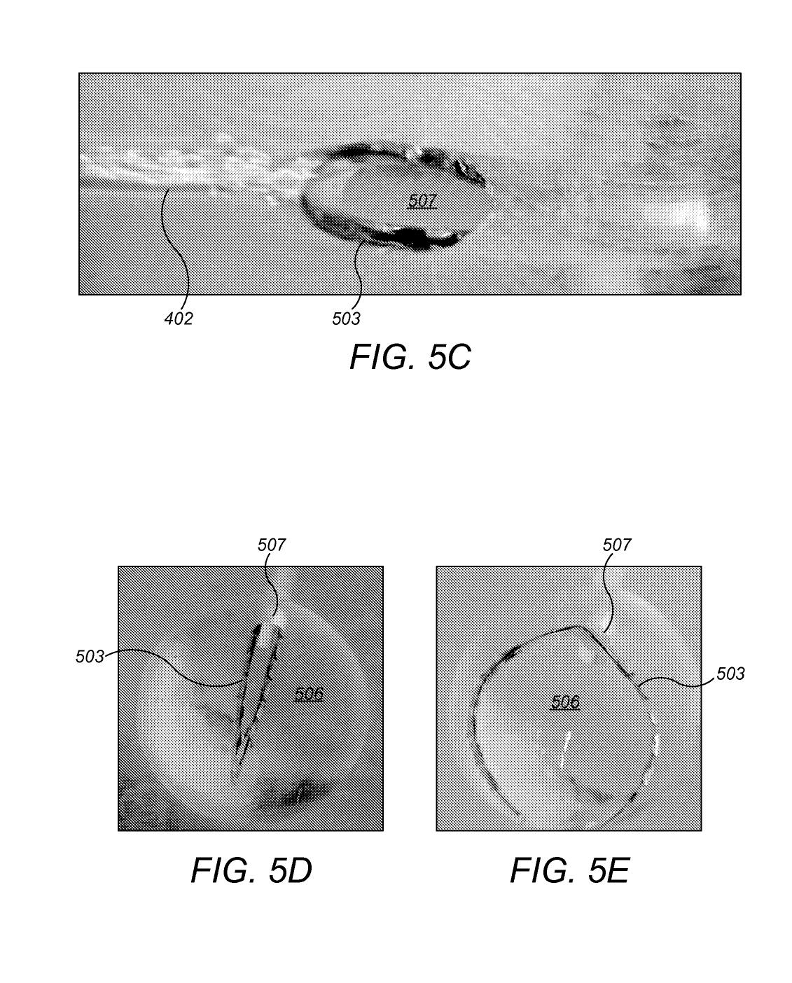

FIG. 5 A shows an alternative embodiment of a common cavity electrode 500 that is foldable into an inverted umbrella shape, with four array branches 503 lying along the center axis forward of the array trunk 402 and the stimulation contacts 504 on the outer surfaces of the array branches 503. FIG. 5 B shows a similar embodiment of a common cavity electrode 500 with two opposing array branches 503 lying along the center axis forward of the array trunk 402 and the stimulation contacts 504 on the outer surfaces of the array branches 503. A springy nitinol support wire 505 is embedded within the opposing array branches 503 as shown. In a specific embodiment, the array branches 503 might typically have a diameter of 0.3 mm and the internal support wire might have a diameter of 0.04 mm. To insert the common cavity electrode 500 into the cochlea, the array branches 503 are folded inward towards the center axis and the distal ends pushed through the single cochleostomy opening 507, as shown in FIG. 5 C. As shown in FIG. 5 D, once the array branches 503 are fully within the cochlear cavity 506, the support rod springs them outward towards the outer wall of the cochlear cavity 506 where the stimulation contacts will be closely adjacent to the neural tissue in the outer wall.

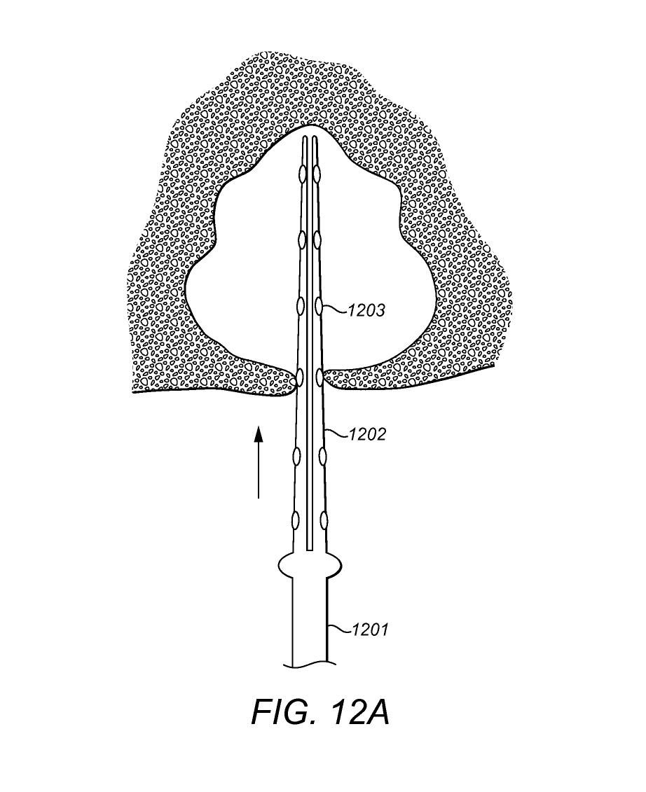

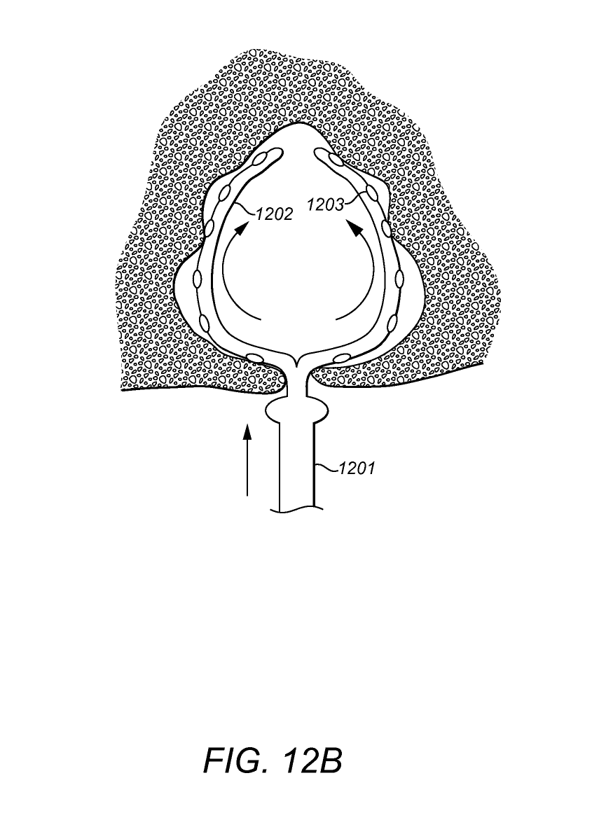

FIG. 12 A-B shows a related embodiment where there are two array branches 1202 which together are about the same size and shape as a conventional cochlear implant electrode array. The array branches 1202 are separated by a longitudinal slit between them along the center axis while the extra-cochlear array trunk 1201 is a single larger branch. The array branches 1202 are advanced through the single cochleostomy opening into the cochlea, and once their distal ends reach the opposite cavity wall, continued pushing naturally bends the array branches 1202 out towards the outer walls of the cochlear cavity. When the array branches 1202 are fully inserted, the stimulation contacts 1203 will lie adjacent to the neural tissues in the outer walls of the cochlear cavity.

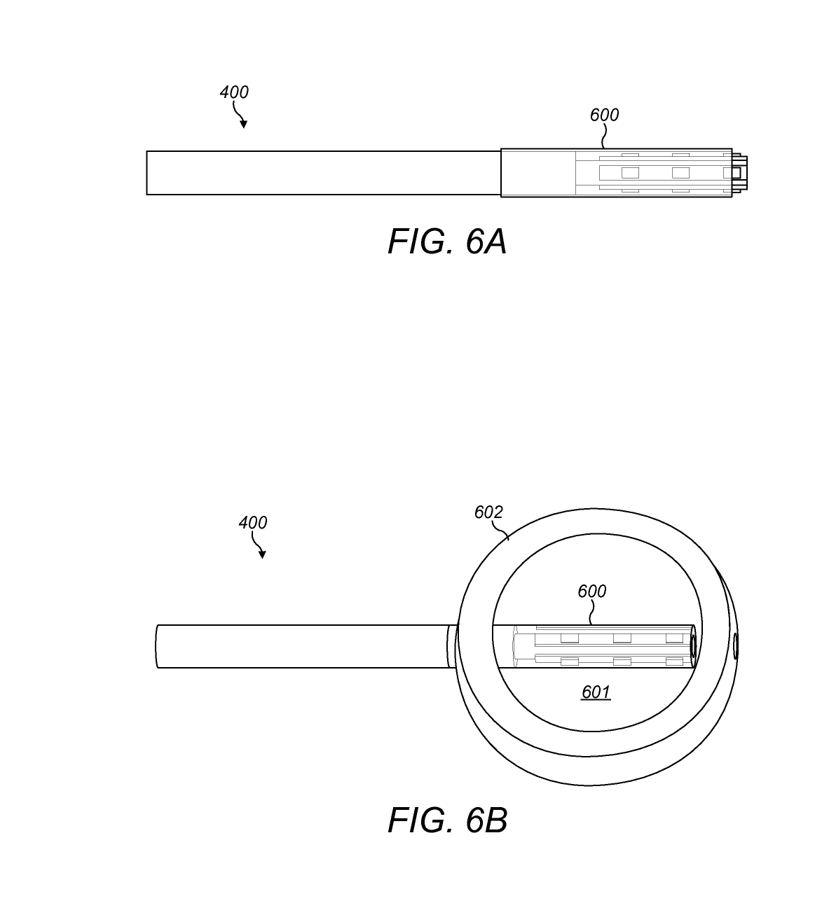

FIG. 6 A shows a common cavity electrode 400 with the array branches 403 folded against the array trunk 402 and fitted within an outer insertion tube 600 made of bio-compatible polymer material for insertion through a single cochleostomy opening in the outer surface of the cavity wall 602 and into the internal cavity of the cochlea 601 as shown in FIG. 6B. In some embodiments the insertion tube 600 may contain a longitudinal slit along its outer surface to remove it from the electrode lead 401 after retraction back outside the cochlea 601. The insertion tube 600 also may include one or depth indicator marks along its outer surface to help the surgeon determine when the electrode array has been fully inserted. Alternatively or in addition, array trunk 402 may include such indicator mark(s).

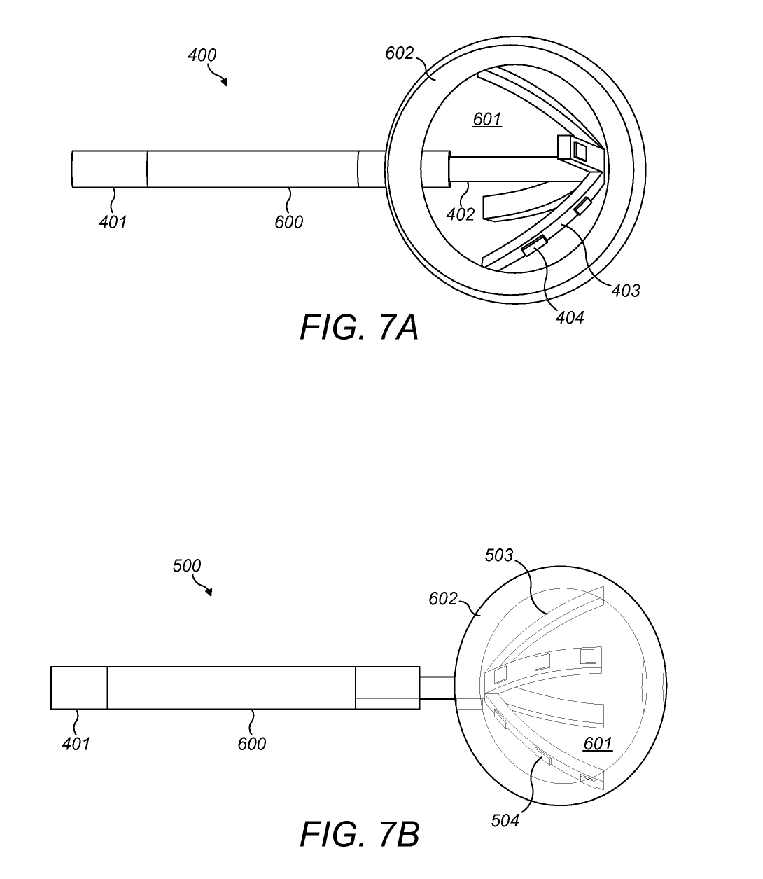

After insertion and retraction of the insertion tube 600, the umbrella-shaped array branches 403 unfold back away from the array trunk 402, as shown in FIG. 7 A to lie within the internal cavity 601 with their outer surfaces against the outer cavity wall 602 to deliver the electrical stimulation signals through the array branch stimulation contacts 404 to adjacent neural tissue for auditory perception by the patient. FIG. 7B shows the same arrangement for an inverted umbrella shape where the array branches 503 unfold within the internal cavity 601 away from the center axis of the electrode array to place the stimulation contacts 504 adjacent to the outer cavity wall 602.

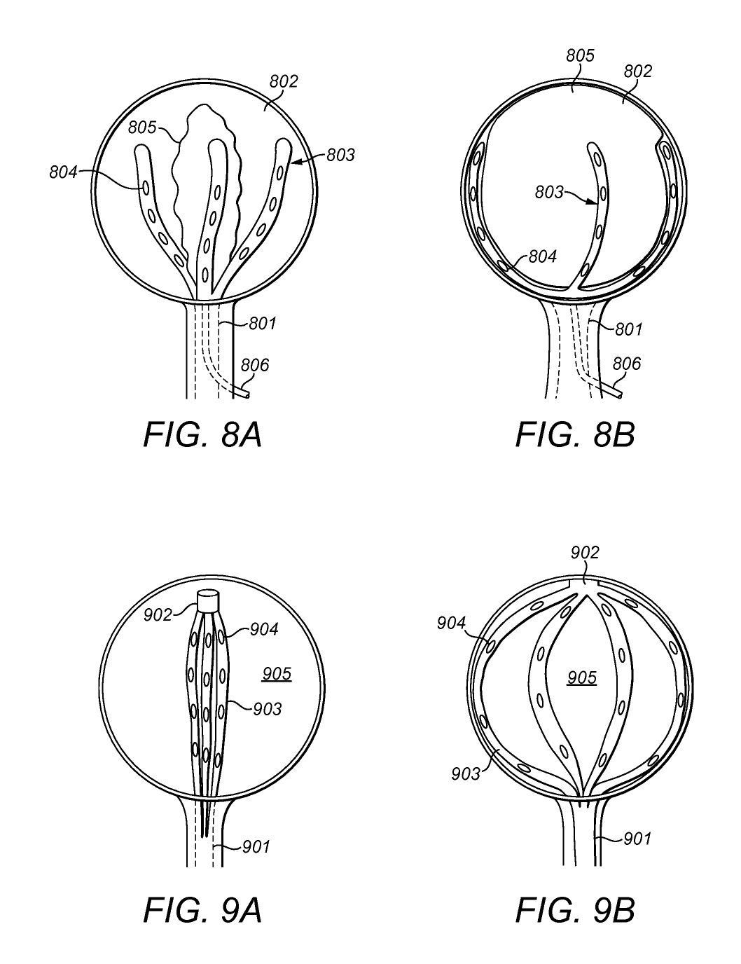

FIG. 8 A-B shows another embodiment of a common cavity electrode. After the array branches 803 have been inserted through the cochleostomy opening into the cochlea 802, an inflation line 806 inflates an expansion balloon 805 that gently pushes the array branches 803 out in a controlled manner until the stimulation contacts 804 lie against the neural tissues at the outer cavity wall.

The inflation balloon 805 may be made of resilient silicone material and/or some or all of the inflation balloon may be made of biodegradable material. Air, gas or biocompatible liquid may be used to fill the inflation balloon 805. In some embodiments, therapeutic substances may be added to the inflation fluid which may be released after surgery (e.g., by decomposition of the biodegradable elements of the balloon) to help the cochlear tissues heal. The amount of inflation fluid needed to inflate the inflation balloon 805 may be determined prior to the implantation surgery, for example, by medical imaging.

The inflation line 806 may be an internal lumen within the electrode lead 801 and/or the expansion balloon 805 may provide a lasting connection between the array branches 803 after the balloon has been inflated. In that case, a sealing mechanism such as a self-sealing membrane should be provided to tightly close the inflation line 806 after the inflation balloon is filled in order to prevent the ingress of bacteria into the cochlea 802. Or the inflation line 806 and expansion balloon 805 may be separate elements from the rest of the electrode. In the latter case, it may be possible to deflate the expansion balloon 805 after the array branches 803 have been deployed, and withdraw the expansion balloon 805 and inflation line 806 from the cochlea 802.

FIG. 9 A-B shows another embodiment of a common cavity electrode with a silicone connector tip 902 at the distal end of the array branches 903. During implantation, the electrode is pushed through the cochleostomy opening into the interior common cavity of the cochlea 905 until the connector tip 902 touches the outer wall of the cavity opposite the cochleostomy opening. The surgeon continues to push the electrode lead 901 towards the cochleostomy opening causing the array branches 903 to bend outward towards the outer cavity wall of the cochlea 905 until the stimulation contacts 904 lie against the neural tissues at the outer cavity wall.

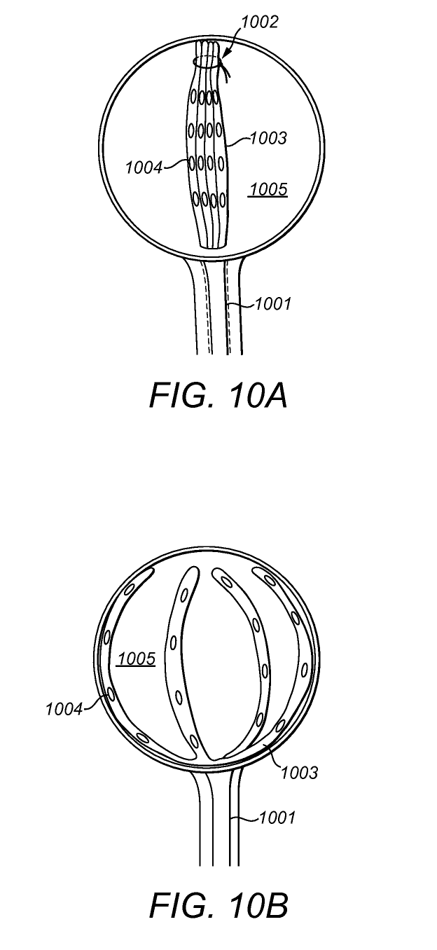

FIG. 10 A-B shows a related embodiment with a temporary tip connection 1002 that allows the array branches 1003 to bend out towards the outer cavity wall of the cochlea 1005 until the stimulation contacts 1004 lie against the neural tissues there. The tip connection 1002 may be a biodegradable wire or filament (e.g., dissolving suture material) which dissolves after a period of time following surgery. Or the tip connection 1002 may be loose enough around the distal tips of the array branches 1003 that it can slip off or be untied after insertion through the cochleostomy opening.

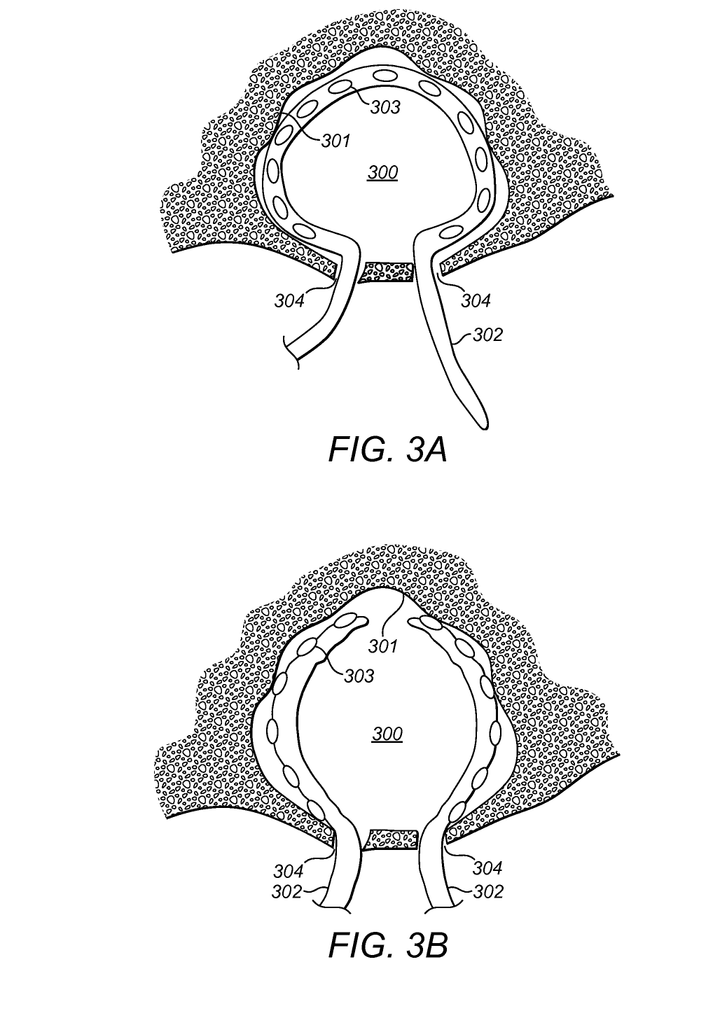

The common electrode arrangements described above cover the distribution of neural structures in a common cavity much better than conventional existing electrodes (e.g., as shown in FIGS. 3 A-B). The common cavity is a three-dimensional volume whereas the existing conventional electrodes of the type shown in FIGS. 3A-B are basically two-dimensional structures. By contrast, the common cavity electrodes described above can better cover all three dimensions of the common cavity.

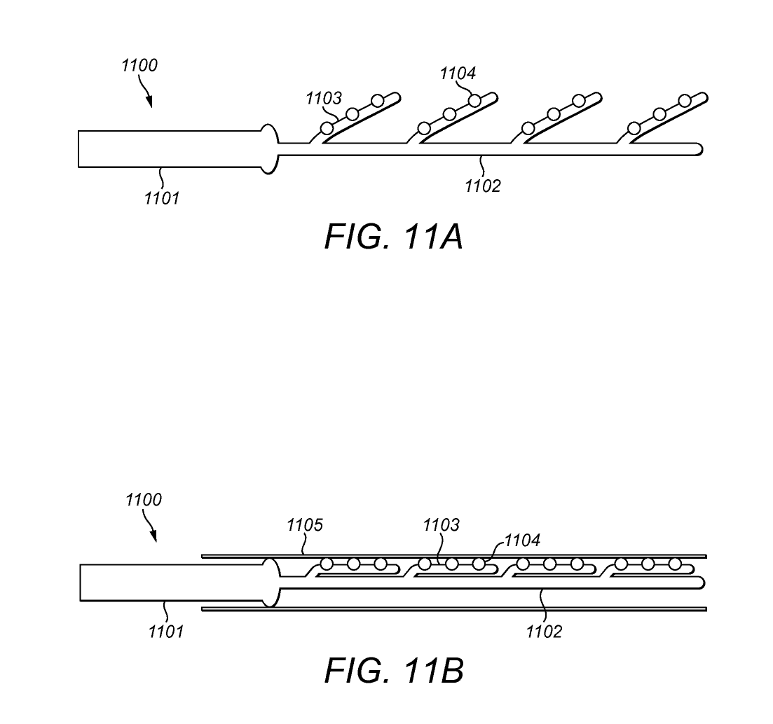

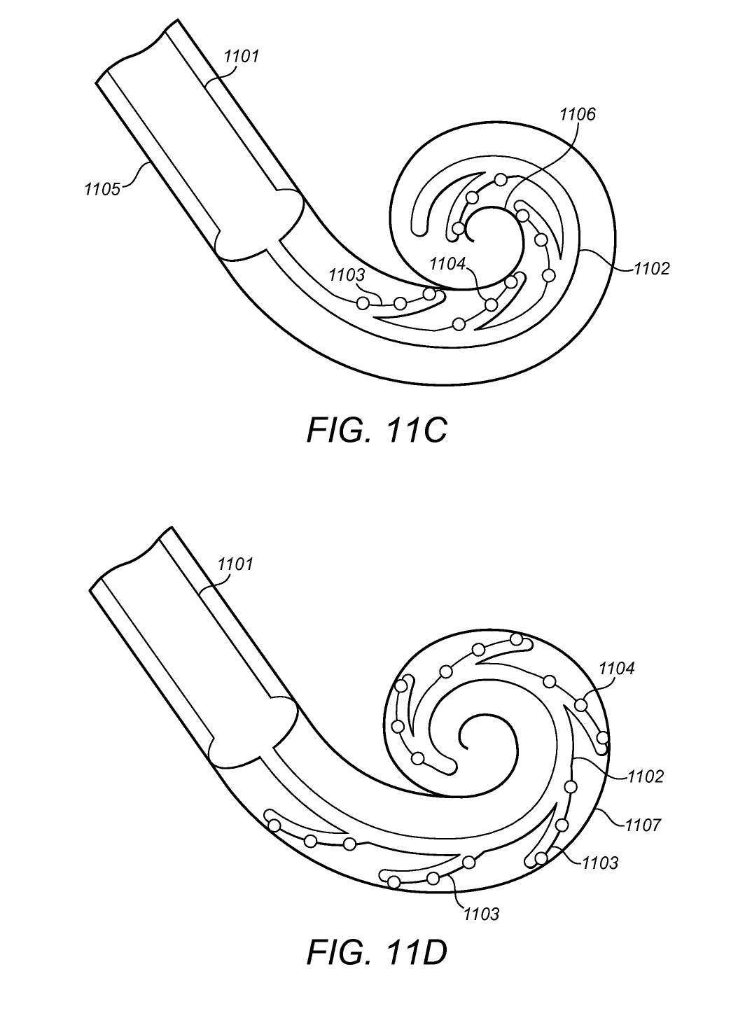

The approach of foldable array branches as described above may also be useful in a cochlear implant electrode for insertion into a conventionally spiral-shaped cochlea or cochlea with incomplete partition, such as in Mondini's dysplasia. In Mondini's dysplasia, the cochlea and the vestibular organ is separated, but in the cochlea, only the basal turn is normally formed leaving the middle and the apical turn to appear as a single cyst, resulting in an incomplete partition. For example, FIGS. 11 A-D show an embodiment of a cochlear implant electrode 1100 for a normal spiral-shaped cochlear anatomy (FIG. 11C) or a cochlea anatomy with incomplete partition (FIG. 11D) with foldable array branches 1103 that lie against the array trunk 1102 during implantation surgery. As shown in FIG. 11B, this may be by means of an outer insertion tube 1105 that fits over the folded array branches 1103 and array trunk 1102 for insertion into the cochlea. Once the array has been fully inserted, the insertion tube 1105 is slid back out of the cochlea over the extra-cochlear electrode lead 1101, allowing the array branches 1103 to fold outward to place the stimulation contacts against the inner modiolar wall 1106 to gently engage the neural tissues there, as shown in FIG. 11C, or against the outer modiolar wall 1107 to engage the neural tissues there, as shown in FIG. 11D. Alternatively, the array branches 1103 may be included on both sides of the array trunk 1102, so that the stimulation contacts 1004 may engage both the outer and inner walls 1106, 1107. The array branches 1103, whether positioned with the array branches 1103 facing the outer modiolar wall, the inner modiolar wall, or both, may be fixed to the array trunk 1102 at either their proximal end (such as shown in FIG. 11C) or distal ends (such as shown in FIG. 11D). If the electrode 1100 is inserted and then slightly retracted, the array branches 1103 may separate more easily when the array branches are fixed at their distal ends, as shown in FIG. 11D.

FIGS. 12 A-B show another embodiment of a common cavity electrode that includes a double branch electrode having electrode contacts 1203 on the individual branches that are preferably arranged at the lateral side of the electrode arrays 1202. This arrangement supports the idea of having a single cochleostomy. In this embodiment, the electrode arrays 1202 are inserted through the cochleostomy into the common cavity until a point opposite the cochleostomy, as shown in FIG. 12A, the two branches of the electrode arrays 1202 will then move apart from each other and contact the wall of the cochlea, as shown in FIG. 12B. During the insertion process of the common cavity electrode, an insertion tube, such as described above, may be used and positioned around the double branch arrangement during the first phase of insertion. When the tips of the electrode arrays 1202 touch the cochlea opposite the cochleostomy, the insertion tube may be retracted in a coordinated manner together with further insertion of the electrode arrangement.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable electrode for a cochlear implant patient with cochlea having a single internal cavity defined by an outer cavity wall, the electrode comprising:
an extra-cochlear electrode lead containing a plurality of signal wires for conducting electrical stimulation signals; and
an intra-cochlear electrode array configured to be inserted into the cochlea through a single cochleostomy opening, the electrode array including a plurality of outer array branches closable about a center axis and each having an outer surface with one or more stimulation contacts for delivering the electrical stimulation signals to adjacent neural tissue;
wherein the electrode array is configured to be closed into a single tube including the array branches for insertion through the single cochleostomy opening into the internal cavity of the cochlea, and
wherein the electrode array is configured to be opened within the internal cavity after insertion into the cochlea so that the array branches move away from the center axis to lie with their outer surfaces against the outer cavity wall to deliver the electrical stimulation signals through the stimulation contacts to adjacent neural tissue for auditory perception by the patient.

2. The implantable electrode according to claim 1, further comprising:
an outer insertion tube configured to contain the closed electrode array for insertion into the cochlea, and retractable back through the cochleostomy opening after insertion of the electrode array into the cochlea to allow the array branches to open within the internal cavity of the cochlea.

3. The implantable electrode according to claim 2, wherein the insertion tube contains a longitudinal slit along its outer surface for removal of the insertion tube from the electrode lead after retraction.

4. The implantable electrode according to claim 2, wherein the insertion tube includes one or depth indicator marks along its outer surface.

5. The implantable electrode according to claim 1, wherein the electrode array is configured to be closed into a closed umbrella shape with the array branches lying along the center axis for insertion into the cochlea and to be opened into an open umbrella shape after insertion into the cochlea.

6. The implantable electrode according to claim 1, wherein the electrode array is configured to be closed into a closed inverted umbrella shape with the array branches lying along the center axis for insertion into the cochlea and to be opened into an open inverted umbrella shape after insertion into the cochlea.

7. The implantable electrode according to claim 6, further comprising:
a springy support wire within the array branches that biases the array branches open.

8. The implantable electrode according to claim 1, wherein the outer surface of each array branch includes a plurality of stimulation contacts.

9. The implantable electrode according to claim 8, wherein each array branch has stimulation contacts on both inner and outer surfaces.

10. The implantable electrode according to claim 1, wherein the electrode array is made of radio-opaque material.

11. The implantable electrode according to claim 10, wherein the array branches have different radio-opaque patterns.

12. The implantable electrode according to claim 10, wherein a distal tip of the electrode array has a radio indicator mark.

13. The implantable electrode according to claim 1, wherein the electrode array includes a central array trunk containing the signal wires against which the array branches are closed for insertion into the cochlea.

14. The implantable electrode according to claim 1, wherein the electrode array includes a central inflation balloon against which the array branches are closed for insertion into the cochlea, wherein the inflation balloon is configured to be filled and expand outward after insertion into the cochlea to move the array branches away from the center axis to place their outer surfaces against the outer cavity wall.

15. The implantable electrode according to claim 14, wherein the inflation balloon is permanently connected to the array branches.

16. The implantable electrode according to claim 14, wherein the inflation balloon is configured to be deflated after being filled and be removed from the cochlea via the single cochleostomy opening.

17. The implantable electrode according to claim 1, wherein the electrode array includes a connector tip configured to hold together distal ends of the array branches, and wherein the electrode array is configured for the array branches to move away from the center axis after the connector tip touches the outer cavity wall opposite the single cochleostomy opening during insertion of the electrode array into the cochlea.

18. The implantable electrode according to claim 17, wherein the connector tip is permanently connected to the distal ends of the array branches.

19. The implantable electrode according to claim 17, wherein the connector tip is configured to be removed from the distal ends of the array branches after insertion of the electrode array into the cochlea.

20. A cochlear implant system having an implantable electrode according to claim 1.

21. A cochlear implant electrode for a cochlear implant patient with an incomplete partition or conventional spiral-shaped cochlea, the electrode comprising:
an extra-cochlear electrode lead containing a plurality of signal wires for conducting electrical stimulation signals; and
an intra-cochlear electrode array configured to be inserted into the cochlea through a single cochleostomy opening, the electrode array including a plurality of outer array branches closable about a center axis and each having an outer surface with one or more stimulation contacts for delivering the electrical stimulation signals to adjacent neural tissue;
wherein the electrode array is configured to be closed into a single tube including the array branches for insertion through the single cochleostomy opening into the internal chamber of the cochlea, and
wherein the electrode array is configured to be opened within the cochlea after insertion so that the array branches move away from the center axis to lie with their outer surfaces against an modiolar wall of the cochlea to deliver the electrical stimulation signals through the stimulation contacts to adjacent neural tissue for auditory perception by the patient.

22. A cochlear implant system having an implantable electrode according to claim 21.
